(19) United States
(12) Patent Application Publication
(10) Pub. No.: US 2006/0095109 A1
(43) Pub. Date: May 4, 2006

(54) Flexible electronic acupuncture device and manufacture method thereof

(76) Inventors: Chen Ming-Daw, Hsinchu City (TW); Huang Jih-Fon, Zhubei City (TW)

Correspondence Address: BRUCE H. TROXELL, SUITE 1404, 5205 LEESBURG PIKE, FALLS CHURCH, VA, 22041, US

(21) Appl. No.: 11/023,566

(22) Filed: Dec. 29, 2004

(30) Foreign Application Priority Data
Oct. 29, 2004 (TW) 93132856

(51) Int. Cl. A61N 1/00; A61N 1/34

(52) U.S. Cl. 607/148

(57) ABSTRACT

A flexible electronic acupuncture device comprises a metal substrate, an insulating layer disposed on the metal substrate, an circuit device disposed on the insulating layer and electrically connected to the metal substrate via a lead through the insulating layer, a rechargeable power module coupled to the circuit device to provide power therefore, a control device coupled to the circuit device to select the output wave form of the flexible electronic acupuncture device, and a skin pad disposed on the metal substrate to contact skin.

### 1. FIELD OF THE INVENTION

The invention is related to a flexible electronic acupuncture device and a manufacture method thereof, and more particularly, to a flexible electronic acupuncture device capable of providing various muscle stimulations by selecting different voltages and frequencies.

### 2. BACKGROUND OF THE INVENTION

Traditional Chinese medicine has been developed for a very long period of time. However, many treatments exercised by traditional Chinese medicine are still remain in mist. Only recently that traditional Chinese medicine has inspired interests of Western scientists and a variety of electronic devices designed to aid the western physician in the practice of this ancient art. One of such traditional Chinese medical treatments is acupuncture. Traditionally, points in the human body (herein called acupuncture points) were reached by piercing the body with fine wires or needles that is now being replaced by electronic acupuncture devices.

Electronic acupuncture, which is also known as high potential therapy apparatus, is a means of using minute electronic pulses to stimulate the body's own healing system. Instead of an invasive needle being placed into the flesh, a simple probe is placed on the surface of the skin and a series of pulses is transmitted through the skin so as to create an electrical field around the human body and acting on the hypothalamus, the hypophysis and even every cell for arousing activity of every cell by way of skin, nerve and nerve fluid so as to enable every organ in the human body to maintain normal functions.

The electronic acupuncture is capable of stimulating pancreas cells to regulate the function of the endocrine system such that it can enable more than 60% of diabetics to maintain in normal blood sugar level. The electronic acupuncture acts on every cell for enabling the same to maintain normal function and electricity balance such that the blood pressure is regulated. It also acts on mitochondria to activate signal transmit system and release ATP energy, reduce products of incomplete metabolism such as leukotrienes and cure atopic dermatitis, asthma, arthritis and rheumatoid arthritis. High potential therapy apparatus can increase calcium ion concentration of serum and reduce phosphorous ion concentration to reach normal calcium/phosphorous ratio so as to maintain blood weak basic. It is helpful for gout, because the uric acid crystal can solute in blood and is thus easily excreted. Acid blood is viscous and narrows vessel, and it is the main cause resulting in cardiovascular diseases. High potential therapy apparatus can make blood basic, reduce free fatty acid, reduce low lipoprotein and increase high lipoprotein. It can, therefore, soften vessel and reduce blood viscosity. High potential therapy apparatus has high voltage and low current output, and it is thus not harmful to human.

A conventional flexible acupuncture device is disclosed in US patent published application no. 20010039444A1, entitles “ELECT-RODE FOR MUSCLE STIMULATION”. Referring to FIG. 1, the device comprises an electrode unit 100, a pad unit 101, a skin pad 102 and a connector 103. The electrode unit 100 is composed of a carrier 1001 and a thin and elastic electrode 1002 attached to the carrier 1001. The skin pad 102 is attached to a side of the carrier 1001 opposite to the electrode 1002. The via holes 104 are punched through the electrode 100 and the skin pad 102 for receiving the connector 103 that can clamp the tray-like carrier 1001 onto the pad carrier 1011. In addition, the connector 103 also serves as the connecting terminal for the stimulating current.

Another flexible acupuncture device is disclosed in US patent published application no. 20040088036A1, entitles “DEVICE FOR SURFACE STIMULATION OF ACUPUNTURE POINTS”. It is a two-layer disc-shaped device. The upper layer 200 is an insulated transparent polymer layer of 3-7 mil in thickness. The lower layer 201 is insulated polyethylene layer of 32 mil in thickness having a side thereof for attaching to the skin, and another side thereof for attaching to the upper layer 200. An electrically conductive gel 202 overfills a central hole through the lower layer 201 to project from the lowermost (in use), skin-contacting surface thereof. Further gel 203 overfills a series of further holes through the lower layer 201 spaced about a circle centered on the hole for the central gel 201. A suitable gel comprises a conductive hydrogel polymer are employed as it is provided for skin contact. a silver/silver chloride film (not shown) sandwiched between the upper layer 200 and the lower layer 201 electrically connects the conductive gel 202 and 203 to the terminals 204 and 205 where an external power can be inputted therefrom while the connection point 206 is connected to a power suuply.

The described conventional flexible electronic acupuncture devices have the drawbacks listed as following:

(1) The conventional flexible electronic acupuncture device requires to be connected to an external power supply and control interface that is inconvenient and not portable.

(2) The structure of the conventional flexible electronic acupuncture device is so complicated that the manufacturing cost is high.

(3) A timer must be added to the control interface for controlling the operating time of the conventional acupuncture device that further increase the manufacturing cost.

### SUMMARY OF THE INVENTION

It is the primary object of the invention to provide a flexible electronic acupuncture device and a manufacture method thereof, capable of integrating a power supply and a control interface therein for enhancing portability and convenience of usage.

It is the secondary object of the invention to provide a flexible electronic acupuncture device and a manufacture method thereof, which is composed of simplified mechanical structure and circuit such that the manufacturing cost of the device is reduced.

Another object of the invention is to provide a flexible electronic acupuncture device and a manufacture method thereof, having a control interface capable of selectively adding a timer for controlling the operating time of the device with respect to the requirements, so that the device has comparatively more design flexibility.

To achieve the above object, a flexible electronic acupuncture device is provided according to a preferred embodiment of the invention, comprising a metal substrate, an insulating layer, an circuit device, a rechargeable power module, a control device and a skin pad. The insulating layer is disposed on the metal substrate. The circuit device is disposed on the insulating layer and electrically connected to the metal substrate via a lead through the insulating layer. The rechargeable power module is coupled to the circuit device so as to provide power to the acupuncture device. The control device is coupled to the circuit device to select the output waveform of the flexible electronic acupuncture device. The skin pad is disposed on the metal substrate for skin contacting.

The present invention also provides a manufacture method of the flexible electronic acupuncture device, the method comprising the steps of: providing a metal substrate while forming a insulating layer thereon; forming an circuit device on the insulating layer; patterning the insulating layer to form a via hole; forming a control device coupled to the circuit device; filling the via hole with silver paste which is solidified to form a lead; connecting a rechargeable power module to the circuit device; and assembling a skin pad to a housing.

A detailed description is given in the following embodiments with reference to the accompanying drawings.

### BRIEF DESCRIPTION OF THE DRAWINGS

The present invention can be more fully understood by reading the subsequent detailed description and examples with references made to the accompanying drawings, wherein:

FIG. 1 is a schematic view of a conventional flexible electronic acupuncture device;

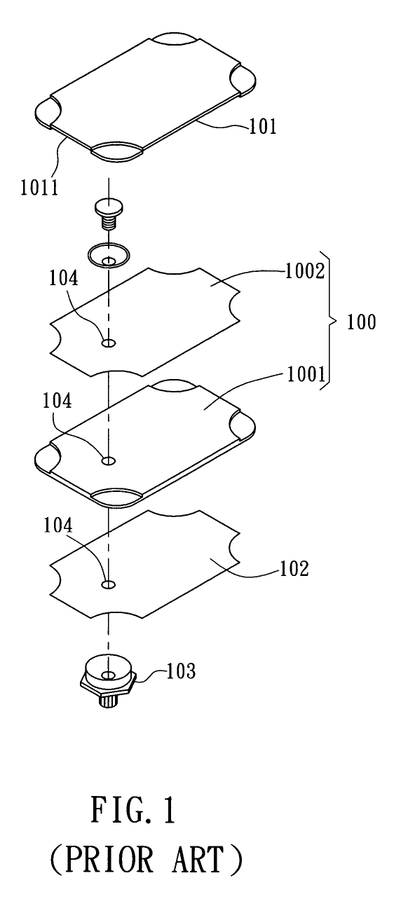

FIG. 2 is another schematic view of a conventional flexible electronic acupuncture device;

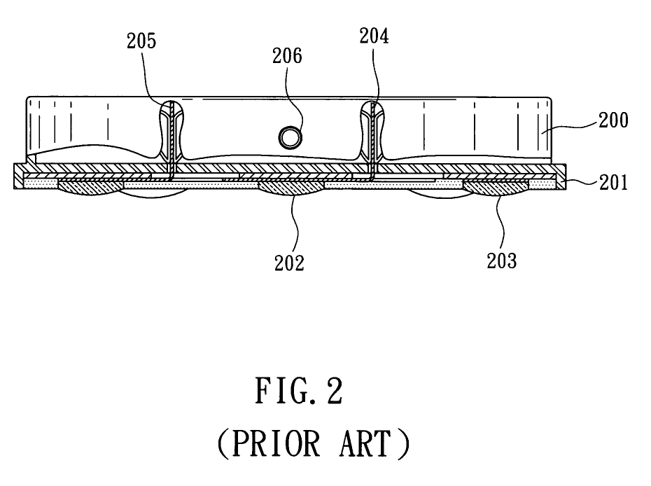

FIG. 3A is a top view of a flexible electronic acupuncture device according to the invention;

FIG. 3B is a cross section of a flexible electronic acupuncture device of the invention;

FIG. 3C is a cross section of a circuit device of a flexible electronic acupuncture device of the invention; and

FIG. 4 is a flowchart of a manufacture method of a flexible electronic acupuncture device according to the invention.

### DESCRIPTION OF THE PREFERRED EMBODIMENT

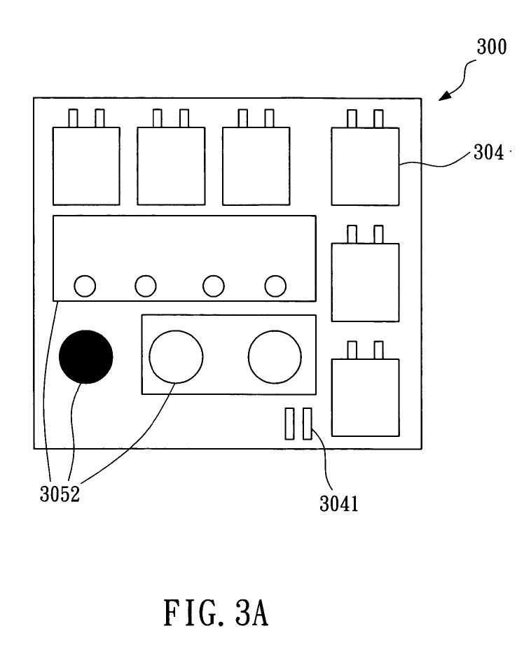

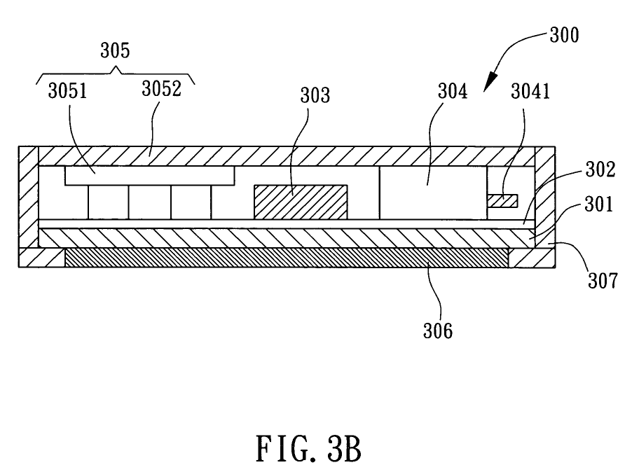

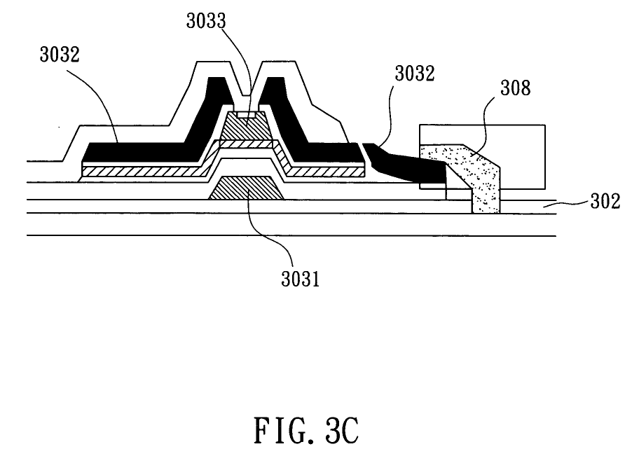

Referring to FIGS. 3A, 3B and 3C, which are respectively a top view and a cross section view of a flexible electronic acupuncture device according to the invention, and a cross section of a circuit device of a flexible electronic acupuncture device according to the present invention. As seen, a flexible electronic acupuncture device 300 of the invention comprises a metal substrate 301, an insulating layer 302, an circuit device 303, a rechargeable power module 304, a control device 305, a skin pad 306 and a housing 307. The insulating layer 302 typically made of silicon nitride is disposed on the metal substrate 301 for insulating the circuit device 303 from the metal substrate 301. The metal substrate 301 is a flat metal plate made of aluminum or copper. The circuit device 303 is disposed on the insulating layer 302 and electrically connected with the metal substrate 301 via a lead 308 extending through the insulating layer 302. The lead 308 can be made of silver paste. A via hole on the insulating layer 302 is formed by lithography etching.

In general, the circuit device 303 is a transistor comprising a gate line 3031, a signal line 3032 and a control gate 3033, which is electrically connected to the metal layer 301 via the lead 308. The circuit device 303 is mainly used to control output waveform of the flexible electronic acupuncture 300 with respect to various voltages and frequencies such that various stimulations can be generated for creating different effects, such as knocking, kneading, pressing and shaking etc. The effects, manufacture method or material of other structures of the flexible electronic acupuncture device 300, such as a protection layer or insulating layer, are well known by those who are skillful in the art, and are omitted.

The rechargeable power module 304 is coupled to circuit device 303 to provide power for the flexible electronic acupuncture device 300. A charging end 3041 is provided for the rechargeable power module 304 to connect to an external power supply and recharge therefrom. The control device 305 is coupled to the circuit device 303 for setting the output waveform of the flexible electronic acupuncture device 300, which controls the selection of turning on/off the flexible electronic acupuncture device 300 and the mode selecting of acupuncture intensity and acupuncture time. The control device 305 comprises a metal sheet 3051 and a set of button 3052, wherein the button set 3052 is connected to the metal sheet 3051. The acupuncture intensity and time modes are selected by pressing the corresponding button of the button set 3052, and the flexible electronic acupuncture device 300 outputs the specified mode accordingly. The flexible skin pad 306 disposed on the metal substrate 301 contacts user's skin. The housing 307 envelops the described elements.

As the rechargeable power module 304 can be a dry cell, a Ni—H battery or a lithium battery, a predetermined storage volume can be preset. That is, since the flexible electronic acupuncture device 300 will stop working while power of the rechargeable power module 304 runs out, the rechargeable power module 304 with a preset volume can be used as a timing control for controlling the acupuncture time of the flexible electronic acupuncture device 300 such that a timing control mode can be void from the device 300 for cost saving.

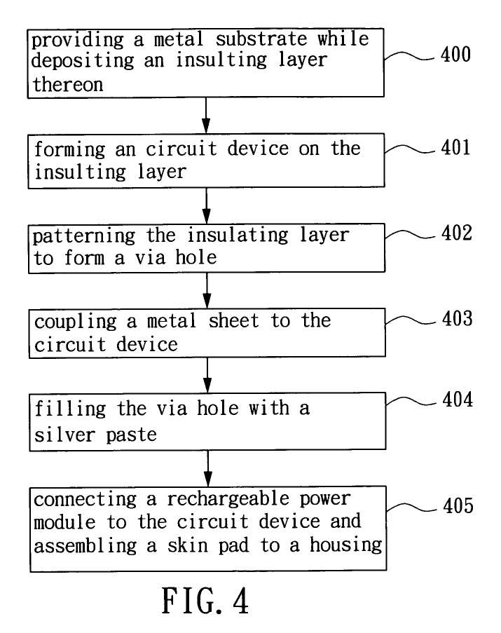

As seen in FIG. 4, the manufacture method of the flexible electronic acupuncture device comprises the steps of:

providing a metal substrate while depositing an insulting layer thereon as seen in step 400, wherein the metal substrate can be polished in advanced to obtain a very flat metal substrate, and the insulating layer can be made of silicon nitride for insulating the metal substrate from certain posterior circuits according to a preferred embodiment of the invention;

forming an circuit device as seen in step 401, wherein a transistor accomplished by a semiconductor process including attaching glass substrate and plating silicon is employed as the circuit device for controlling the output waveform of the flexible electronic acupuncture device with respect to various voltages and frequencies such that various stimulations can be generated for creating different effects, such as knocking, kneading, pressing and shaking etc;

patterning the insulating layer to form a via hole as seen in step 402, wherein the insulating layer is patterned by lithography etching to form a via hole for electrical connecting the circuit device with the metal substrate;

coupling a metal sheet to the circuit device as seen in step 403, wherein a circuit are formed on a metal sheet by low temperature poly silicon (LTPS) process while the metal sheet is coupled to the circuit device, moreover, a button set is arranged with respect to the metal sheet and serves as a control device corresponding to the circuit device for controlling the output waveform;

filling the via hole with silver paste as seen in step 404, wherein the silver paste is filled into the via hole and baked to solid for forming a lead therein, and thus the lead extends through the insulating layer for electrically connecting the circuit device and the metal substrate, such that enable the acupuncture device of the invention to be manufactured with simplified circuit and mechanical structure and thus greatly reduced the manufacturing cost thereof by using a simplified semiconductor process;

connecting a rechargeable power module to the circuit device and assembling a skin pad to a housing, wherein the rechargeable power module is connected to the circuit device to provide power for the flexible electronic acupuncture device, and the rechargeable power module is connected to an external power supply via a charging end, in addition, the rechargeable power module can be a dry cell, a Ni—H battery or a lithium battery, such that a simple and portable flexible electronic acupuncture device without external power supply is accomplished.

To sum up, a power supply and a control interface are effectively integrated into the flexible electronic acupuncture device of the invention. Thus, the flexible electronic acupuncture device with simple circuit device and mechanical structure is portable and easily to use. The described embodiment is not to limit the scope of the invention. Those who are skillful in the art can make some modifications such as different batteries, different circuit devices and different positions of the charging end.

While the preferred embodiment of the invention has been set forth for the purpose of disclosure, modifications of the disclosed embodiment of the invention as well as other embodiments thereof may occur to those skilled in the art. Accordingly, the appended claims are intended to cover all embodiments which do not depart from the spirit and scope of the invention.

What is claimed is:

1. A flexible electronic acupuncture device, comprising:
a metal substrate;
an insulating layer, disposed on the metal substrate;
an circuit device, disposed on the insulating layer and electrically connected to the metal substrate via a lead passing through the insulating layer;
a rechargeable power module, coupled to the circuit device for providing power for the electronic acupuncture device;
a control device, coupled to the circuit device for controlling the output waveform of the flexible electronic acupuncture device; and
a skin pad, disposed on the metal substrate and adapted for skin contacting.

2. The flexible electronic acupuncture device as claimed in claim 1, wherein the insulating layer is made of silicon nitride.

3. The flexible electronic acupuncture device as claimed in claim 1, wherein the circuit device is a transistor.

4. The flexible electronic acupuncture device as claimed in claim 1, wherein the lead is made of silver paste.

5. The flexible electronic acupuncture device as claimed in claim 1, wherein the rechargeable power module is one selected from the group consisting of a dry cell, a Ni—H battery and a lithium battery.

6. The flexible electronic acupuncture device as claimed in claim 1, wherein the acupuncture device further comprises a housing.

7. The flexible electronic acupuncture device as claimed in claim 1, wherein the control device further comprises a metal sheet and a button set connected to the metal sheet.

8. A method of manufacturing a flexible electronic acupuncture device, comprising the steps of:
providing a metal substrate while forming an insulating layer on the metal substrate;
forming a circuit device on the insulating layer;
patterning the insulating layer to form a via hole;
forming a control device coupled to the circuit device;
filling the via hole with silver paste while the silver paste is baked to solid for forming a lead;
connecting a rechargeable power module to the circuit device; and
assembling a skin pad to a housing.

9. The method of manufacturing a flexible electronic acupuncture device as claimed in claim 8, wherein the insulating layer is made of silicon nitride.

10. The method of manufacturing a flexible electronic acupuncture device as claimed in claim 8, wherein the circuit device is a transistor.

11. The method of manufacturing a flexible electronic acupuncture device as claimed in claim 8, wherein the rechargeable power module is one selected from the group consisting of a dry cell, a Ni—H battery and a lithium battery.

12. The method of manufacturing a flexible electronic acupuncture device as claimed in claim 8, wherein the control device further comprises a metal sheet and a button set connected to the metal sheet.
